(19) United States
(12) Patent Application Publication
(10) Pub. No.: US 2013/0316462 A1
(43) Pub. Date: Nov. 28, 2013

(54) RAPID AND HIGH-THROUGHPUT ANALYSIS OF STEROLS/STANOLS OR DERIVATIVES THEREOF

(71) Applicant: HEALTH DIAGNOSTIC LABORATORY, INC., Richmond, VA (US)

(72) Inventors: James L. BRUTON, JR., Henrico, VA (US); Alexandra SHERMAN, Richmond, VA (US)

(73) Assignee: HEALTH DIAGNOSTIC LABORATORY, INC., Richmond, VA (US)

(21) Appl. No.: 13/797,560

(22) Filed: Mar. 12, 2013

Related U.S. Application Data
(60) Provisional application No. 61/651,982, filed on May 25, 2012.
(60) Provisional application No. 61/696,613, filed on Sep. 4, 2012.

(51) Int. Cl. G01N 33/92

(52) U.S. Cl. CPC G01N 33/92
USPC 436/71

(57) ABSTRACT

This invention relates to a rapid, high-throughput process for analyzing one or more sterols/stanols or derivatives thereof in a plurality of samples. The method comprises the steps of introducing a plurality of samples containing one or more sterols/stanols or derivatives thereof into individual vessels in a multi-vessel plate; cleaving the one or more sterols/stanols or derivatives thereof of each sample in the multi-vessel plate to form free sterols/stanols; extracting the free sterols/stanols of each sample by solid phase extraction; and detecting the level of the extracted free sterols/stanols in each sample by liquid chromatography tandem mass spectrometry. In this process, the free sterols/stanols do not undergo an additional derivitization step of adding a functional group to the free sterols/stanols prior to the detecting step.

### CROSS-REFERENCE TO RELATED APPLICATIONS

This application claims the benefit of priority to U.S. Provisional Patent Application Ser. No. 61/651,982, filed May 25, 2012; and U.S. Provisional Patent Application Ser. No. 61/696,613, filed Sep. 4, 2012; both of which are hereby incorporated by reference in their entirety.

### FIELD OF THE INVENTION

This invention relates to a rapid, high-throughput process for analyzing one or more sterols/stanols or derivatives thereof in a plurality of samples.

### BACKGROUND

Sterols are essential components of cell membranes in animals (zoosterols, e.g., cholesterol) and plants (phytosterols). Cholesterol is essential for life, as it is a crucial membrane molecule and the precursor of steroid hormones, vitamin D, and bile acids. People vary in their cholesterol balance—the amount of cholesterol they synthesize, absorb, and excrete. After dietary absorption into the enterocyte, virtually all non-cholesterol sterols and some cholesterol are effluxed back into the gut lumen via membrane sterol efflux transporters. Most humans absorb approximately 50% of the luminal sterols into the enterocyte, but hyperabsorbers absorb 60-80% and hypoabsorbers approximately 20-30%. After absorption, cholesterol, but not phytosterols, can be esterified and incorporated with triglycerides and phospholipids into chylomicrons.

Phytosterols serve no physiologic function in humans or animals, and cannot be synthesized or readily absorbed by humans or animals. Because humans with normal physiology absorb very few phytosterols/stanols, their assay in blood serves as a marker of intestinal absorption. Similarly, cholesterol precursor sterols serve as synthesis biomarkers. Hyperabsorbers, in whom phytosterols do gain systemic entry, are diagnosable by increased absorption markers. With rare loss-of-function mutations in ABCG5 or ABCG8, all phytosterols are absorbed and none are effluxed back out, leading to phytosterolemia, with up to 100-fold elevation in plasma phytosterol levels, associated with childhood xanthomas and premature atherosclerosis. Very high levels of cholestanol, a cholesterol metabolite yet also a marker of absorption, occur in the rare recessive condition cerebrotendinous xanthomatosis (CTX), which are associated with several neurological deficits. Markers of both cholesterol absorption (e.g., beta-sitosterol, campesterol, cholestanol) and cholesterol synthesis (e.g., desmosterol) can be measured and manipulated by drugs such as statins, which can block cholesterol synthesis, or by drugs such as ezetimibe, fenofibrate, supplemental phytosterols or stanols, which can reduce cholesterol absorption.

Most of the inherited disorders of cholesterol metabolism can be diagnosed by noninvasive analysis of the sterol profiles in serum. Moreover, a rapid, accurate evaluation of plasma sterol/stanol levels, particularly the cholesterol absorption and/or synthesis biomarkers can help predict patients' risks of cardiovascular diseases, personalize risk assessment, optimize lipid-lowering lifestyle/drug therapy, and plan a more effective lipoprotein treatment regimen.

Conventional analysis of sterols in serum typically uses gas chromatography (GC) or liquid chromatography (LC) in combination with various detection methods, such as flame ionization detection, electron ionization-mass spectrometry (ELMS), etc. However, these techniques are time-consuming and typically require a laborious pretreatment procedure, such as derivatization, to increase the sensitivity and specificity of sample analysis.

The sample pre-treatment prior to the analysis can be time-consuming and can lower the sensitivity of the sample analysis, if not properly designed. For instance, manual extraction of sterols/stanols from biological samples is a laborious and time-consuming process and can introduce manual errors, and contaminations. Derivatization of sterols/stanols not only introduces a laborious step and increases the time to carry out the process, but can also introduce unnecessary and undesirable toxicity due to the use of the derivatization agent.

Therefore, there is a need in the art to develop a rapid, high throughput technique for improved analysis of sterols/stanols in a plurality of samples with high sensitivity and high accuracy. This invention answers this need.

### SUMMARY OF THE INVENTION

The embodiments of this invention relates to a rapid, high-throughput process for analyzing one or more sterols/stanols or derivatives thereof in a plurality of samples. The method comprises the steps of introducing a plurality of samples containing one or more sterols/stanols or derivatives thereof into individual vessels in a multi-vessel plate; cleaving the one or more sterols/stanols or derivatives thereof of each sample in the multi-vessel plate to form free sterols/stanols; extracting the free sterols/stanols of each sample by solid phase extraction; and detecting the level of the extracted free sterols/stanols in each sample by liquid chromatography tandem mass spectrometry. In this process, the free sterols/stanols do not undergo an additional derivitization step of adding a functional group to the free sterols/stanols prior to the detecting step.

The process described here provides a sensitive, rapid and high throughput method for simultaneous quantification of various sterols/stanols combined with an automated extraction of sterols/stanols from the samples. This process does not require derivatization of sterols/stanols prior to the analysis. The entire process for quantification of various sterols/stanols during the detection step can be carried out in less than about 7 minutes.

During the process, after each step when the sample or reagent is introduced or transferred into the vessel, or during holding, reacting, and/or mixing the samples, each vessel of the multi-vessel plate can be sealed by a matching multi-cap mat to withstand high temperature, or to prevent the sample from evaporation or contamination.

In an exemplary embodiment, this rapid, high-throughput sterol/stanol analysis test measures four non-cholesterol sterols/stanols. β-Sitosterol, campesterol and cholestanol were measured as markers of cholesterol absorption (cholestanol, marker of absorption efficiency and to diagnose Cerebrotendinous Xanthomatosis (CTX)); desmosterol, an intermediary sterol in the formation of cholesterol, was measured as a marker of cholesterol synthesis. The entire process for the quantifications of these four markers during the detection step can be carried out in less than about 7 minutes.

Analyzing sterol/stanol levels in plasma can provide information on whether a patient is more of an absorber or a synthesizer, thus helping the physician personalize a drug therapy and plan a more effective lipoprotein treatment regimen.

Embodiments of the present invention may be used to provide preliminary diagnoses of certain conditions, or to monitor the progression of a condition and/or the efficacy of a therapy being used to treat the condition.

Additional aspects, advantages and features of the invention are set forth in this specification, and in part will become apparent to those skilled in the art on examination of the following, or may be learned by practice of the invention. The inventions disclosed in this application are not limited to any particular set of or combination of aspects, advantages and features. It is contemplated that various combinations of the stated aspects, advantages and features make up the inventions disclosed in this application.

### BRIEF DESCRIPTION OF THE DRAWINGS

FIGS. 1A-1B are photographs showing a machine capable of being used for the automated solid phase extraction, the Hamilton Microlab STAR. FIG. 1A shows the exterior view of Hamilton Microlab STAR, and FIG. 1B shows the interior view of Hamilton Microlab STAR.

FIG. 2 is a photograph showing AB Sciex Model 5500 Mass Spectrometer with Shimadzu Prominence Pumps, a suitable LC-MS/MS system.

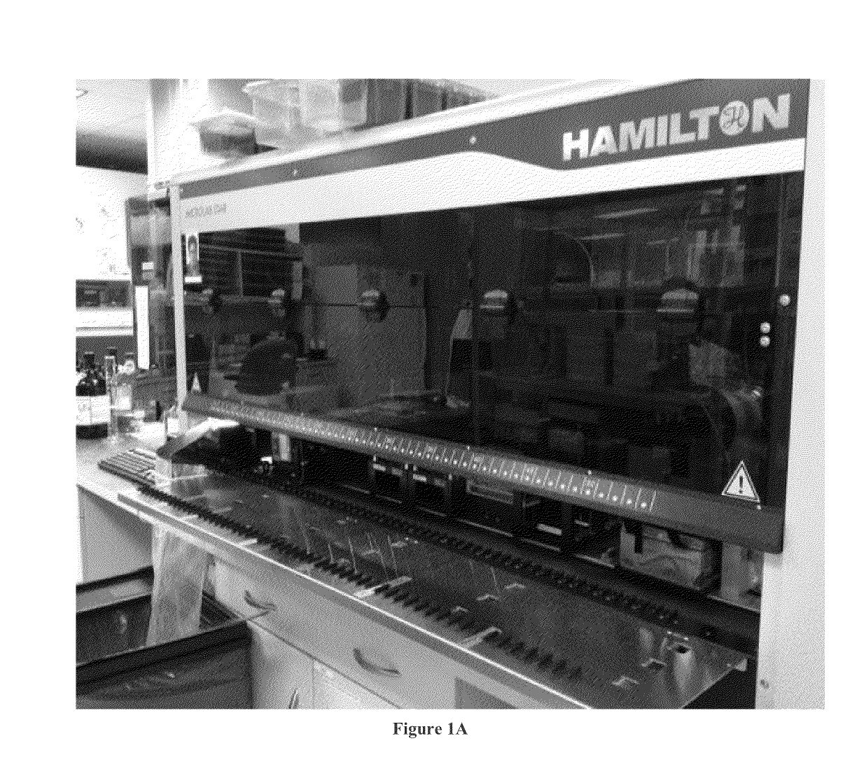

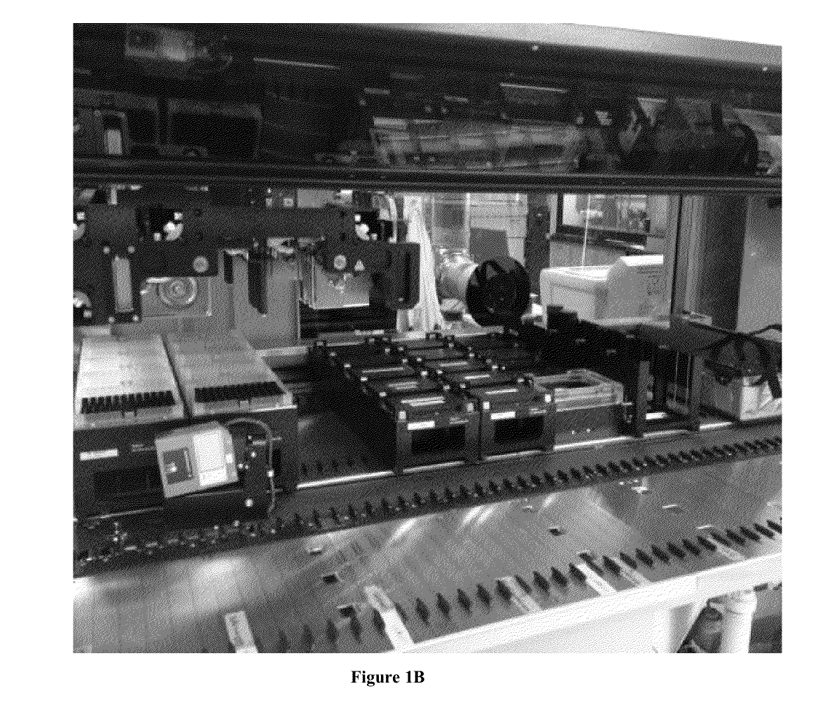

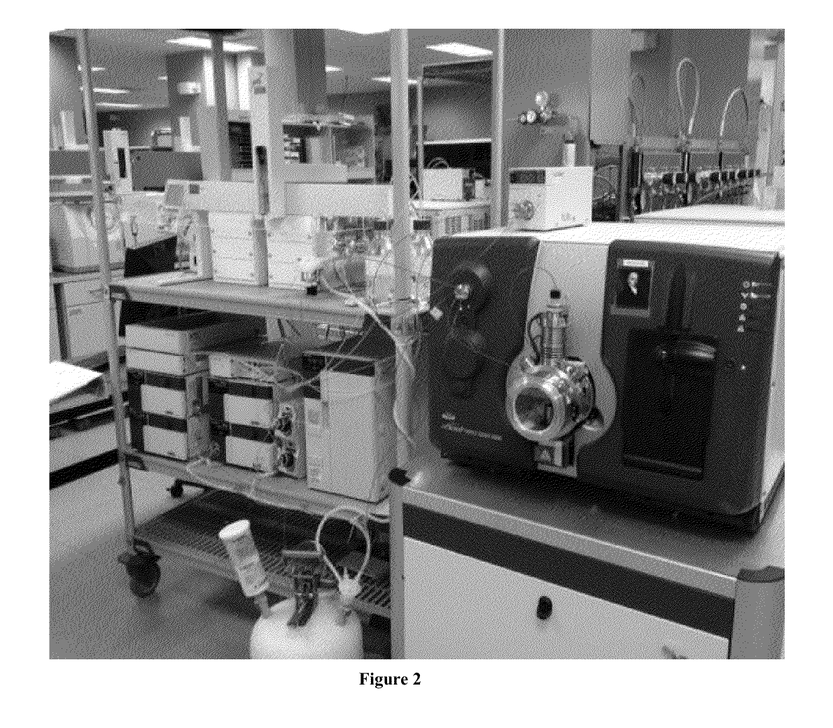

### DETAILED DESCRIPTION OF THE INVENTION

This invention relates to a rapid, high-throughput process for analyzing one or more sterols/stanols or derivatives thereof in a plurality of samples. The process employs a system or an apparatus that enables automated, high-throughput conduction of one or more steps of the process.

This system/apparatus can include at least one multi-vessel plate. Each vessel of the multi-vessel plate is a unit for holding a sample, or mixing and/or reacting a sample with one or more solvents or reagents. Each vessel is wide and tall enough to allow for adequate mixing, and thin enough to allow the multi-vessel plate to fit in an automated fluid handling station and/or an automated multi-vessel plate handling station. The vessel can have a round or flat base depending on the requirement of the system.

The multi-vessel plate can have a matching multi-cap mat that is capable of sealing the vessels of the multi-vessel plate during the holding, mixing and/or reacting the sample. The lining of the multi-cap mat which contacts the tops of the vessels in the multi-vessel plate is made of a material that does not deteriote and does not contaminate the vessel when heating to the desirable temperature. For instance, the material can be teflon.

Optionally, a multi-vessel plate holder that has a matching size with the multi-vessel plate can be used to hold the multi-vessel plate for temporary storage, or during the holding, mixing and/or reacting the sample. The multi-vessel plate holder has sealing units, whereby the multi-vessel plate holder, when the sealing units are engaged, can press the matching multi-cap mat onto the tops of the vessels in the multi-vessel plate sealing the vessels, so as to withstand high pressure and high temperature conditions.

The system/apparatus can optionally hold a library of stock multi-vessel plates, which can have a variety of functions. For instance, they can be used to contain samples, react with reagents for certain reactions, or for extraction or separation of certain components in the samples, etc. Multi-vessel plates can be created as needed. For instance, to create a multi-vessel solid phase extraction plate, a solid phase extraction column/plate can be placed into each vessel, and an appropriate solvent can be automated pipetted to pre-condition the column/plate for later use.

An automated liquid/fluid handling device (or an automated multi-vessel plate handling device) can be used in the system. This automated liquid handling device can introduce weighed samples and/or reagents into each vessel. For instance, the automated liquid handling device may contain an automated pipetting device that is capable of automatedly pipetting a weighed amount of sample and/or solvent into each vessel.

The automated liquid handling device can also include an element for automated homogenization (e.g., automated shaking, mixing, or vortexing), automated heating/cooling, or simultaneously automated homogenization and heating/cooling. This automated heating/cooling can also be carried out on a separate multi-vessel plate heating/cooling unit. Similarly, the automated homogenization can be carried out on a separate multi-vessel plate shaking/mixing/vortexing unit. Alternatively, the automated heating/cooling and homogenization elements can be combined in a same automated device.

The system/apparatus may further include equipment for labeling vessels in the multi-vessel plate and a label detector. For instance, the labeling equipment can be an automated bar-coding equipment, and the label detector can be an automated bar code detector. The labeling equipment and label detector enable precise mapping the measurements obtained to each sample in the vessel.

The system/apparatus additionally includes a multi-vessel plate measuring unit to analyze the sterols/stanol samples. The measuring unit enables automated quantization of each sterol/stanol in the sample of each vessel. This measuring unit can be of modular construction, thereby permitting the different measuring units to be exchanged depending on the measurement task. Suitable measuring units include chromatography-mass spectrometry devices. For instance, the measuring unit can be a liquid chromatography tandem mass spectrometry (LC-MS/MS).

The system/apparatus can include an integrated robot system having one or more robots or separate robotics transporting the multi-vessel plates/mats/holders from station to station for sample and reagent addition, holding, mixing, incubation, and measurements.

The system/apparatus can also include data processing and control software. By means of an intelligent software program, the analysis of a plurality of samples may be optimized in terms of time, by conducting different steps in parallel when operating on batches of multi-vessel plates.

In one aspect, the process comprises the steps of introducing a plurality of samples containing one or more sterols/stanols or derivatives thereof into individual vessels in a multi-vessel plate; cleaving the one or more sterols/stanols or derivatives thereof of each sample in the multi-vessel plate to form free sterols/stanols; extracting the free sterols/stanols of each sample by solid phase extraction; and detecting the level of the extracted free sterols/stanols in each sample by liquid chromatography tandem mass spectrometry. In this process, the free sterols/stanols do not undergo an additional derivitization step of adding a functional group to the free sterols/stanols prior to the detecting step.

Sterols include zoosterols and phytosterols. The predominant zoosterol is cholesterol. Production of cholesterol depends on its cellular synthesis (all cells) and absorption (enterocytes). Some of the intermediary sterols in the synthetic chain are squalene, lathosterol and desmo sterol, measurements of which can serve as a marker of cholesterol synthesis. Sterols that have structural similarity to cholesterol are also referred to as non-cholesterol sterols. The human diet includes many exogenous sterols from plants (e.g., sitosterol, campesterol, and stigmasterol), animals (e.g., cholesterol), shellfish sources (e.g., desmosterol, and fucosterol) and yeast sources.

There are over forty different plant sterols (or phytosterols). Phytosterols are similar in structure to cholesterol, but have methyl, ethyl or other groups in their aliphatic side chains. These differences minimize their absorption compared to cholesterol. Sitosterol represents 80% of non-cholesterol sterols in the diet.

Each of these sterols, as well as others known to one skilled in the art, falls under the definition of “sterol” for the purposes of this invention.

Stanols are simply saturated sterols. For instance, the stanol metabolite of cholesterol is called cholestanol; and the stanol metabolite of sitosterol is sitostanol.

Each of these stanols, as well as others known to one skilled in the art, falls under the definition of “sterol” for the purposes of this invention.

The process provides for a rapid, high throughput, automated determination of the levels of any one or more sterols/stanols or derivatives in a large assembly of samples. Exemplary sterols/stanols markers to be analyzed include, but are not limited to, desmosterol, campesterol, cholestanol, β-sitosterol, squalene, lathosterol, stigmasterol and/or fucosterol. Desmosterol, campesterol, cholestanol, and β-sitosterol are the typical cholesterol synthesis and absorption biomarkers analyzed in the process.

The process may be used to analyze sterols/stanols from any biological sample containing sterols/stanols or derivatives thereof to be analyzed. The biological sample can be a blood component such as plasma, serum, red blood cells, whole blood, platelets, white blood cells, or mixtures thereof. The sterols/stanols to be analyzed may exist in the biological sample as free sterols/stanols or any forms derived from the sterols/stanols (e.g., a sterol/stanol ester) during a biological process.

In one embodiment, the step of introducing a plurality of samples containing one or more sterols/stanols or derivatives thereof into individual vessels in a multi-vessel plate is carried out by pipetting a weighed amount of each sample into each vessel.

Sterols/stanols may exist as various forms in the biological sample, such as sterol/stanol esters, sterol glycosides, acylated sterol glycosides, etc. Before analyzing the sterols/stanols in the biological sample, free sterols/stanols may be cleaved from the sterols/stanols derivatives, if present.

In one embodiment, the step of cleaving the one or more sterols/stanols or derivatives thereof of each sample comprises pipetting a cleaving agent into each sample in the multi-vessel plate; vortexing the composition containing the sample and cleaving agent in each vessel; and heating the multi-vessel plate to a desirable temperature. These steps can be carried out with an automated liquid handling device, automated homogenization (e.g., automated shaking, mixing, or vortexing), or automated heating device, or a device enables simultaneously automated homogenization and heating, as described herein.

Typically, the cleavage step involves hydrolyzing the sterol/stanol derivatives. For instance, the cleavage step can involve saponification of ester of sterol/stanol (i.e., base hydrolysis of sterol/stanol esters). The saponification reaction typically takes place in presence of an alkali hydroxide or alkaline hydroxide catalyst, such as sodium hydroxide or potassium hydroxide. The alkali hydroxide or alkaline hydroxide catalyst may be dissolved in a solvent, such as ethanol or methanol. The temperature for the saponification reaction typically ranges from about 40 to about 50° C.

The free sterols/stanols is further extracted or separated from the other components in the samples by solid-phase extraction (SPE). Typically, a commercially available pre-packed polymer or glass mini disposable columns (cartridges) or plates can be used.

In one embodiment, the extracting step comprises transferring each sample in the multi-vessel plate to a multi-vessel solid phase extraction plate after the cleaving step; and eluting the free sterols of each sample from the multi-vessel solid phase extraction plate into a multi-vessel collecting plate. These steps can be carried out with an automated liquid handling device, as described herein.

During the SPE process, the sample can be passed through SPE columns/plates, with or without applying pressure. The sterols/stanols retained on the stationary phase of SPE can be removed from the stationary phase by using an appropriate eluent. The eluted sterols/stanols are then collected for further analysis. Exemplary eluent includes dicholoride methane, methanol, or acetonitrile. For example, when one or more of desmosterol, campesterol, cholestanol, and β-sitosterol are being analyzed, dicholoride methane can be used for a good recovery of the analytes.

The extracting step may further comprise drying the eluted free sterols/stanols of each sample in a multi-vessel collecting plate; and adding a reconstitution solution to the dried free sterols/stanols in the multi-vessel collecting plate to reconstitute the free sterols/stanols, prior to the sterol/stanol analysis. Exemplary reconstitution solution includes methanol/isopropanol/formic acid solution or methanol/acetonitrile solution. For instance, reconstitution solution can be 80:20 methanol:isopropanol in 0.1% formic acid, or 50:50 methanol:acetonitrile.

A detailed description of the extraction and separation of free sterols/stanols is shown in Example 1.

The extracted sterols/stanols can be detected by chromatography-mass spectrometry. For instance, liquid chromatography tandem mass spectrometry (LC-MS/MS) may be utilized to analyze various sterols/stanols contained in plasma and serum with a single test.

For quantitative analysis of sterols/stanols in the sample, an internal standard can be added to each sample in the multi-vessel plate. The internal standard is used for calibration, for instance, by plotting the ratio of the sterol/stanol sample signal to the internal standard signal as a function of the analyte concentration present in the standards. For instance, the internal standard can be cholesterol or a cholesterol derivative, such as cholesteryl stearate, ketocholesterol, etc. The internal standard can be a deuterated internal standard. When a deuterated internal standard is used, the deuterated internal standard can be a deuterated form of any one or more of the sterols/stanols to be abalyzed. For example, one or more of d6-desmosterol, d7-campesterol and d7-β-sitosterol can be used as internal standards when desmosterol, campesterol, and sitosterol are being analyzed. An internal standard can be added after the sterol/stanol sample is introduced to the multi-vessel plate, prior to the cleaving step, prior to the extracting step or prior to the detecting step. Typically, the internal standard is added immediately after the sample is introduced to the multi-vessel plate. The addition of an internal standard can be carried out with an automated liquid handling device, as described herein.

### EXAMPLES

The following examples are given as particular embodiments of the invention and to demonstrate the practice and advantages thereof. It is to be understood that the examples are given by way of illustration and are not intended to limit the specification or the claims that follow in any manner.

### Example 1

### Method of High-Throughput Analysis of Sterols/Stanols

All the steps of the process were performed on the Hamilton Microlab STAR unless otherwise noted.

To 150 μL of sample (plasma or serum), 50 μL of deuterated internal standard was added. After thorough mixing, 1 mL of 2% potassium hydroxide in ethanol was added and the samples underwent a saponification reaction for 30 minutes at 45° C.

A Plexa Bond Elut solid phase extraction (SPE) plate was pre-conditioned with 500 μL of methanol, followed by 500 μL of HPLC grade water. Next, each sample was cleaned with 1 mL HPLC grade water and then applied to the SPE plate. The samples were pulled through the SPE plate using positive pressure. Then, the samples were eluted from the SPE plate into a sample collection plate using 500 μL methylene chloride. The plate was then removed from the Hamilton, and placed onto the Biotage® SPE Dry at 60° C. for approximately 30 minutes. The plate was then returned to the Hamilton, and samples were reconstituted with 200 μL of 80:20 methanol:isopropanol 0.1% formic Acid.

The resulting samples were then injected onto an AB Sciex 5500 MS/MS. The specific transitions monitored were m/z 367/161 for desmosterol; m/z 383/147 for campesterol; m/z 371/95 for cholestanol; m/z 397/147 for β-sitosterol; m/z 373/161 for d₆-desmosterol; m/z 390/161 for d₇-campesterol; and m/z 404/161 for d₇-β-sitosterol. The samples were not derivatized and the total run time per sample was 7 minutes.

### Example 2

### Methods for Automated Sample Preparation and Fast LC-MS/MS Analysis

The following exemplary procedures have been programmed in Hamilton Microlab STAR system to illustrate the sterol/stanol sample pre-treatment and detections using the automated system/apparatus including the multi-vessel plates with matching multi-cap mats, automated liquid handling devices, automated labeling equipment and a label detector, automated SPE device, automated multi-vessel plate measuring unit, and the data processing and control software, as described in the above embodiments.

Pre-Incubation Sample Preparation
1. Place sample aliquot tubes into 32 position Hamilton sample carrier in deck positions 13-15. Place empty tubes in the spaces for blanks
2. Print batch bar code from “Bartender” software on Hamilton desktop:
   a. Choose “Plate_BC_Sterols_Hamilton”.
   b. Last label printed will show up, double-click on batch number to change it.
   c. Change number under “screen data”, click OK.
3. Go to File, Print, and fill in “Number of serialized labels” to print more than one batch at a time.
4. Put the barcode on the front of a 96 position MicroLiter plate with 2.5 mL glass inserts (hereby referred to as “96 well glass insert plate”).
5. Open Microlab Star Run icon on Hamilton desktop.
6. Go to File, Open, “Sterols_V1.1”
7. Click on green arrow at top.
8. Fill in how many samples are in the batch. Click OK.
9. If barcode error: pull sample carrier out, fix barcode, push back in place, click Repeat, click Execute.
10. If still error, manually enter barcode information.
11. Monitor Hamilton method while it is running to address any errors. Follow any and all prompts.
12. Reload tips. Replace any sets that are not full. In the software, the number has to be typed in manually:
   SLIM tips Channels, cut (300 uL) 96. Click OK.
   SLIM tips 96CO-RE, uncut (300 uL) 288. Click OK.
   1000 μL filtered tips 672. Click OK.
13. Load the samples and 96 well glass insert plate onto the appropriate position on the Hamilton deck:
   a. 32 position sample carriers, lanes 13-15 on Hamilton deck
   b. 96 well glass insert plate, lane 38 on Hamilton deck, position 4 on carrier: Hamilton will pipette 150 uL of standard, quality control or sample (plasma or serum) into the 96 well glass insert plate
14. Once the sample transfer has finished, the Hamilton program prompts “Pour IS reagent into the correct reagent plate.” The Internal Standard working solution should be in position 5 of plate carrier F in lane 19 of the Hamilton deck. Hamilton then pipettes 50 μL of internal standard to each insert in the plate.
15. Hamilton prompts “Cover glass tube tray and move to heater/shaker. Click OK.” Hamilton prompts “Make sure plate is sitting properly on the shaker and secure Velcro straps. Click OK.” Hamilton vortexes the plate.
16. Once vortexing is finished, Hamilton prompts “Remove velcro straps and cover. Place glass tube tray back in original position. Click OK.”
17. Hamilton prompts “Pour 2% KOH reagent into proper reagent plate.” The 2% KOH reagent should be placed in position 5 of plate carrier G in lane 25 of the Hamilton deck. Hamilton pipettes 1 mL of reagent to each insert in the plate.
18. Hamilton prompts “Cover glass tube tray and vortex for 1 minute. Place back on the Hamilton in the correct position.”
19. Once vortex is finished, Hamilton prompts “Place into 45 degree water bath for 30 minutes.”

Solid Phase Extraction Preparation:
20. Once incubation is finished, remove the plate from the water bath and sit on bench top to cool during SPE plate preparation.
21. Hamilton prompts “Place a SPE plate on top of a waste collection tray and put in the proper position. Fill correct reagent plates with MeOH and H2O. Then press OK.” The SPE plate and waste collection tray should be in position 4 of carrier I in lane 38 of the Hamilton deck. The MeOH reagent plate should be in position 4 of carrier F in lane 19 of the Hamilton deck. The H₂O reagent plate should be in position 4 of carrier G in lane 25 of the Hamilton deck.
22. Hamilton adds 500 μl of MeOH to the SPE plate. Hamilton pipettes 500 μL of MeOH to the SPE plate, and then prompts “Move SPE plate and waste plate to positive pressure manifold. Apply pressure at no greater than 6 psig for 30 seconds or until all reagent is through the plate. When finished, place the SPE plate and the waste plate back into the correct position on the Hamilton and click OK.”
23. Hamilton adds 500 μL of H2O and then will prompt “Move SPE plate and waste plate to positive pressure manifold. Apply pressure at no greater than 6 psig for 30 seconds or until all reagent is through the plate. When finished, place the SPE plate and the waste plate back into the correct position on the Hamilton and click OK.”

Post-Incubation Sample Preparation:
24. Hamilton prompts “Remove the cover from the glass tube tray and refill H₂O reagent trough if necessary.” and then pipettes 500 μL of H2O into each of the sample inserts.
25. Hamilton prompts “Cover glass tube tray and vortex for 1 minute. Place back on the Hamilton in the correct position and click OK.”
26. Hamilton transfers samples from glass inserts to SPE plate.
27. Hamilton prompts “Remove SPE plate and waste collection tray from Hamilton and place on positive pressure manifold. Apply pressure at no greater than 6 psig for 30 seconds or until all samples are through the plate.”
28. Hamilton prompts “Replace waste collection plate with final sample plate in the carrier and return SPE to Hamilton deck. Press OK.” The final sample plate should be in position 4 of carrier I, under the SPE plate in lane 38 on the Hamilton deck.
29. Hamilton prompts “Pour Dichloromethane into correct reagent plate.” Dichloromethane should be in position 3 on carrier F, in lane 19 on the Hamilton deck
30. Hamilton pipettes 500 μL of dichloromethane into the SPE plate and then will prompt “Remove SPE plate and final sample tray from Hamilton and place on positive pressure manifold. Apply pressure at no greater than 6 psig for 30 seconds or until all samples are through the plate.”
31. Hamilton prompts “Remove deep well collection plate from positive pressure manifold and place on the Biotage SPE Dry for evaporation.” After 20 minutes, Hamilton prompts “Cover plate with parafilm and place in refrigerator for 5 min or until cool.” Perform this step only when samples have dried down completely (may take longer than 20 minutes). Hamilton prompts “Pour reconstitution solution into correct reagent plate. Place dry plate in correct position on the Hamilton for addition of recon solution.” Perform this step only when plate is cool to the touch. Dry plate should be in position 2 on carrier F in lane 19 and reconstitution solution should be in position 3 on carrier G in lane 25 on the Hamilton deck.
32. Hamilton adds 200 μL of recon solution to each well of the dry sample plate and then prompt “Cover plate and move to heater shaker.” Hamilton vortexes final sample plate.
33. Locate the Mapping file created by Hamilton. Open the file and copy the standard, QC and specimen ID numbers.

LC-MS/MS Method

| | |
|---|---|
| Comment: | Sterols_Poro_Stream_1 |
| Synchronization Mode: | LC Sync |
| Auto-Equilibration: | Off |
| Acquisition Duration: | 3 min 0 sec |
| Number Of Scans: | 571 |
| Periods In File: | 1 |
| Acquisition Module: | Acquisition Method |
| Software version | Analyst 1.5.2 |

MS Method Properties:

| Period 1: | |
|---|---|
| Scans in Period: | 571 |
| Relative Start Time: | 0.00 msec |
| Experiments in Period: | 1 |
| Period 1 Experiment 1: | |
| Scan Type: | MRM (MRM) |
| Scheduled MRM: | No |
| Polarity: | Positive |
| Scan Mode: | N/A |
| Ion Source: | Heated Nebulizer |
| Resolution Q1: | Unit |
| Resolution Q3: | Unit |
| Intensity Thres.: | 0.00 cps |
| Settling Time: | 0.0000 msec |
| MR Pause: | 5.0070 msec |
| MCA: | No |
| Step Size: | 0.00 Da |

| | | |
|---|---|---|
| @Q1 Mass (Da) | Q3 Mass (Da) | Dwell(msec) |

Desmosterol

| 367.339 | 161.100 | | 40.00 |
|---|---|---|---|
| | DP | 130.0 | |
| | CE | 60.00 | |
| | CXP | 20.00 | |

Campesterol

| 383.371 | 147.100 | | 40.00 |
|---|---|---|---|
| | DP | 140.00 | |
| | CE | 60.00 | |
| | CXP | 13.00 | |

Cholestanol

| 371.371 | 95.100 | | 40.00 |
|---|---|---|---|
| | DP | 120.00 | |
| | CE | 60.00 | |
| | CXP | 9.00 | |

β-Sitosterol

| 397.707 | 147.100 | | 40.00 |
|---|---|---|---|
| | DP | 140.00 | |
| | CE | 50.00 | |
| | CXP | 14.00 | |

Desmosterol d6

| 373.300 | 161.200 | | 40.00 |
|---|---|---|---|
| | DP | 120.00 | |
| | CE | 20.00 | |
| | CXP | 17.00 | |

Campesterol d7

| 390.400 | 161.100 | | 40.00 |
|---|---|---|---|
| | DP | 120.00 | |
| | CE | 20.00 | |
| | CXP | 17.00 | |

β-Sitosterol d7

| 404.300 | 161.200 | | 40.00 |
|---|---|---|---|
| | DP | 120.00 | |
| | CE | 20.00 | |
| | CXP | 17.00 | |

Parameter Table (Period 1 Experiment 1):

| | |
|---|---|
| CUR: | 40.00 |
| CAD: | 7.00 |
| TEM: | 500.00 |
| GS1: | 50.00 |
| GS2: | 0.00 |
| NC: | 4.00 |
| EP | 10.00 |

Valco Valve Diverter

| | Total Time (min) | Position |
|---|---|---|
| 1 | 0.1 | B |
| 2 | 2.9 | A |

Software Application Properties

| | |
|---|---|
| Display Name: | MPX Driver |

Method Data:

| Stream Options | |
|---|---|
| Inject Sample on Stream Number: | 1 |
| Loading Pump | |
| Loading Pump Flow Rate: | 0 mL/min |
| Sample Equilibration Duration: | 5 sec |
| Sample Equilibration Channel: | A |
| Sample Loading Duration: | 5 sec |
| Sample Loading Channel: | A |
| Sample Handling | |
| Default Injection Volume: | 5 μL |
| Read Barcode: | No |

Gradient Pump
Gradient Table

| | | |
|---|---|---|
| 0 | 97 | 1 |
| 3 | 97 | 1 |
| 3.01 | 100 | 1 |
| 5 | 100 | 1 |
| 5.01 | 97 | 1 |
| 6 | 97 | 1 |

| Column Oven | |
|---|---|
| Oven Set Point: | 40 degrees Celsius |
| Acquisition Window | |
| Start Time: | 1.5 min. |
| End Time: | 4.5 min. |
| Other Options | |
| Post Clean with Solvent 1: | 2 |
| Post Clean with Solvent 2: | 2 |
| Valve Clean with Solvent 1: | 2 |
| Valve Clean with Solvent 2: | 2 |
| Air Volume: | 1 μL |
| Filling Speed: | 5 μL/sec |
| Injection Speed: | 10 μL/sec |
| Error Recovery Policy:: | If any stream has an error |

### Example 3

### Validation of Sterols/Stanols High-Throughput Automated Process

Pre-treatment and high-throughput automated sterols/stanols assay of desmosterol, campesterol, cholestanol, and β-sitosterol were carried out according to the exemplified procedures described in Examples 1 and 2.

Validations of the high-throughput analytical method have been performed in full compliance with the Clinical Laboratory Improvements Amendments of 1988 (CLIA '88) enacted by the Congress and a document entitled “Guidance for Industry Bioanalytical Method Validation,” published by the U.S. Department of Health and Services, Food and Drug Administration (May 2001).

Validation is a useful guidepost when developing and implementing a novel bioanalytical method. Exemplary validations parameters being determined include recovery of analytes in the assay and reproducibility of the recovery, dilution linearity of analytes, precision of the assay (intra-batch and inter-batch precisions), and method comparison between this high-throughput, automated, solid phase extraction sterols/stanols assay and the manual liquid/liquid extraction (with hexane) sterols/stanols assays. The results are shown in Tables 1-4 below.

Recovery of an analyte (e.g., desmosterol, campesterol, cholestanol, or β-sitosterol) in the sterol/stanol assay is the detector response (i.e., LC-MS/MS detector response) obtained from a known amount of the analyte added to and extracted from the sample, compared to the detector response obtained for the true concentration of the analyte. Recovery pertains to the extraction efficiency of an analytical method within the limits of variability.

One unspiked sample pool and three different concentration levels of spiked sample pools were used to complete the spike/recovery tests. The amount of sterols/stanols measured in the unspiked pool was the native amount of sterols/stanols present in the plasma sample. A concentrated spiking solution in methanol containing all four analytes (desmosterol, campesterol, cholestanol, and β-sitosterol) was prepared. This concentrated spiking solution was then added to the plasma pool at three different concentration levels, resulting in spiked pool levels 1, 2 and 3. No more than 2% of this concentrated spiking solution was added to the plasma pool. The concentrated spiking solution was diluted into the analytical range of the assay to determine its actual amount. The amounts of the analytes spiked into the spiked pool levels 1, 2 and 3 were then calculated and compared to the amount measured in the unspiked plasma pool to determine the theoretical amount. The measured amount in each of the spiked pool levels 1, 2 and 3 was then compared to the corresponding theoretical amount in each of the spiked pool levels 1, 2 and 3, obtaining % recovery at the three concentration levels.

Recovery of an analyte is not necessarily 100%, but the extent of recovery of an analyte of a good analytical method should be consistent, precise, and reproducible. Typically, mean recovery of the true concentration of the analyte within 85-115% is an acceptable range of recovery known in the art. Recovery test can demonstrate whether a method measures all or only part of the analyte present. Recovery greater than 100% indicates that the method has a degree of error causing an over-measurement of the analyte, as known in the art. The results of the recovery of the sterols/stanols in this automated sterols/stanols assay and reproducibility of the recovery are shown in Table 1. The results demonstrate that these validation parameters passed the corresponding acceptance criteria. Accordingly, the results confirmed and validated the high-throughput, automated sterols/stanols assay as a viable bioanalytical method.

TABLE 1
Recovery of analytes in the sterol/stanol high-throughput
automated assay^{a}
Spiked into Plasma Pool (μg/mL)

| | Re- | | Re- | | Theo- | |
|---|---|---|---|---|---|---|
| | sult 1 | Result 2 | sult 3 | Mean | retical | Recovery |
| Desmosterol | | | | | | |
| UnSpiked Pool | 0.841 | 0.854 | 0.888 | 0.86 | N/A | N/A |
| Spiked Pool Level 1 | 2.25 | 2.02 | 2.02 | 2.10 | 1.89 | 111.1% |
| Spiked Pool Level 2 | 3.07 | 3.2 | 3.11 | 3.13 | 2.91 | 107.3% |
| Spiked Pool Level 3 | 6.27 | 6.16 | 5.98 | 6.14 | 5.99 | 102.4% |
| Mean Recovery | | | | | | 106.9% |
| Campesterol | | | | | | |
| UnSpiked Pool | 3.68 | 3.79 | 3.73 | 3.73 | N/A | N/A |
| Spiked Pool Level 1 | 4.84 | 4.7 | 4.78 | 4.77 | 4.70 | 101.5% |
| Spiked Pool Level 2 | 6.04 | 5.45 | 5.85 | 5.78 | 5.67 | 101.9% |
| Spiked Pool Level 3 | 8.02 | 8.4 | 8.48 | 8.30 | 8.59 | 96.7% |
| Mean Recovery | | | | | | 100.0% |
| Cholestanol | | | | | | |
| UnSpiked Pool | 3.09 | 3.21 | 3.09 | 3.13 | N/A | N/A |
| Spiked Pool Level 1 | 4.36 | 3.91 | 4.25 | 4.17 | 4.18 | 99.8% |
| Spiked Pool Level 2 | 5.13 | 4.93 | 4.63 | 4.90 | 5.24 | 93.5% |
| Spiked Pool Level 3 | 8.17 | 7.73 | 8.24 | 8.05 | 8.39 | 95.9% |
| Mean Recovery | | | | | | 96.4% |
| β-Sitosterol | | | | | | |
| UnSpiked Pool | 2.47 | 2.5 | 2.54 | 2.50 | N/A | N/A |
| Spiked Pool Level 1 | 3.58 | 3.65 | 3.44 | 3.56 | 4.18 | 85.0% |
| Spiked Pool Level 2 | 4.75 | 3.99 | 4.56 | 4.43 | 5.24 | 84.7% |
| Spiked Pool Level 3 | 7.4 | 7.54 | 7.7 | 7.55 | 8.39 | 89.9% |
| Mean Recovery | | | | | | 86.5% |

^{a}Acceptance criteria: 85-115% mean recovery of theoretical value.

A dilution-linearity experiment provides information about the precision of the assay results for samples tested at different levels of dilution in the chosen sample diluent. Linearity is defined relative to the calculated amount of analyte based on the standard curve. An assay method provides flexibility to assay samples with different levels of analyte, if the dilution linearity is good over a wide range of dilution. The dilutional linearity in the sterol/stanol high-throughput automated assay was processed by a serial dilution (dilution of ×2, ×4, ×8, ×16 times) of the sterol/stanol sample in high plasma with 5% Bovine Serum Albumin, and the results are shown in Table 2. Linearity recovery greater than 100% indicates the method has an error present causing an over measurement of the analyte, as known in the art. The results demonstrate that dilution linearity parameters passed the corresponding acceptance criteria known in the art (i.e., 80-120% mean recovery of theoretical value). Accordingly, the results confirmed and validated the high-throughput, automated sterols/stanols assay as a viable bioanalytical method.

TABLE 2
Dilutional linearity in the sterol/stanol high-throughput automated assay^{a}
Serial Dilution of High Plasma Pool with 5% Bovine Serum Albumin

| | | | | |
|---|---|---|---|---|
| Sample | Results (μg/mL) | Mean | Theoretical | Recovery |

Desmosterol

| | | | | | | |
|---|---|---|---|---|---|---|
| Base | 8.24 | 7.81 | 7.82 | 7.957 | N/A | N/A |
| ×2 | 4.69 | 4.18 | 4.03 | 4.300 | 3.978 | 108.1% |
| ×4 | 2 | 2.05 | 2.15 | 2.067 | 1.989 | 103.9% |
| ×8 | 1.08 | 1.08 | 1.08 | 1.080 | 0.995 | 108.6% |
| ×16 | 0.56 | 0.576 | 0.552 | 0.563 | 0.497 | 113.1% |

Campesterol

| | | | | | | |
|---|---|---|---|---|---|---|
| Base | 7.2 | 7.58 | 7.32 | 7.367 | N/A | N/A |
| ×2 | 3.92 | 3.66 | 3.34 | 3.640 | 3.683 | 98.8% |
| ×4 | 1.83 | 1.79 | 1.72 | 1.780 | 1.842 | 96.7% |
| ×8 | 0.986 | 0.9 | 0.895 | 0.927 | 0.921 | 100.7% |
| ×16 | 0.494 | 0.48 | 0.477 | 0.484 | 0.460 | 105.0% |

Cholestanol

| | | | | | | |
|---|---|---|---|---|---|---|
| Base | 8.01 | 7.93 | 8.09 | 8.010 | N/A | N/A |
| ×2 | 4.14 | 3.3 | 3.44 | 3.627 | 4.005 | 90.6% |
| ×4 | 1.94 | 2.32 | 1.86 | 2.040 | 2.003 | 101.9% |
| ×8 | 1.08 | 0.937 | 0.804 | 0.940 | 1.001 | 93.9% |
| ×16 | 0.457 | 0.489 | 0.489 | 0.478 | 0.501 | 95.5% |

β-Sitosterol

| | | | | | | |
|---|---|---|---|---|---|---|
| Base | 6.89 | 6.95 | 7.05 | 6.963 | N/A | N/A |
| ×2 | 3.66 | 3.46 | 3.82 | 3.647 | 3.482 | 104.7% |
| ×4 | 1.74 | 1.87 | 1.65 | 1.753 | 1.741 | 100.7% |
| ×8 | 0.885 | 0.878 | 0.841 | 0.868 | 0.870 | 99.7% |
| ×16 | 0.482 | 0.489 | 0.485 | 0.485 | 0.435 | 111.5% |

^{a}Acceptance criteria: 80-120% mean recovery of theoretical value.

Precision of an analytical assay describes the closeness of individual measures of an analyte when the assay is applied repeatedly to multiple aliquots of a single homogeneous sample. Precision should be measured using a minimum of five determinations per concentration. The precision determined at each concentration level may not exceed 15% of the coefficient of variation (CV) except for the lower limit of quantification (LLOQ), where it may not exceed 20% of the CV. The precision of the sterol/stanol high-throughput automated assay was assessed to determine the intra-batch and inter-batch precisions, respectively, and the results are shown in Table 3. In the table, Pools 1 and 2 were the lowest calibrator (LLOQ) and highest calibrator (ULOQ), respectively. All calibrators were in 5% Bovine Serum Albumin matrix. Pools 3 and 4 were the quality control materials in serum. Pool 5 was a plasma pool. By these measurements, the precisions in all matrices were evaluated across the entire analytical measurement range of the assay.

The results from Table 3 demonstrate that the precision parameters passed the corresponding acceptance criteria known in the art (i.e., ≦15% for intra-batch (within run); and ≦20% for inter-batch (within lab)). Accordingly, the results confirmed and validated the high-throughput, automated sterols/stanols assay as a viable bioanalytical method.

TABLE 3
Precision of the sterol/stanol high-throughput automated
assay (intra-batch and inter-batch precisions) ^{a}

| | | | | |
|---|---|---|---|---|
| Pool1- | Pool2- | Pool3-QC ^{d} | Pool4-QC | Pool5-Plasma |
| LLOQ ^{b} | ULOQ ^{C} | Low | High | Pool |

Desmoterol μg/mL

| | | | | | |
|---|---|---|---|---|---|
| Mean (μg/mL) | 0.1261 | 10.2712 | 0.9776 | 4.4908 | 1.1524 |
| within run % CV ^{e} | 10.0% | 4.7% | 8.0% | 2.6% | 13.4% |
| within lab % CV | 15.3% | 6.0% | 7.2% | 2.8% | 12.2% |

Campesterol μg/mL

| | | | | | |
|---|---|---|---|---|---|
| Mean (μg/mL) | 0.1190 | 10.4944 | 3.4524 | 6.5616 | 4.7486 |
| within run % CV | 7.3% | 4.6% | 5.3% | 6.2% | 6.3% |
| within lab % CV | 12.9% | 8.8% | 8.1% | 9.0% | 11.0% |

Cholestanol - μg/mL

| | | | | | |
|---|---|---|---|---|---|
| Mean (ug/mL) | 0.0980 | 10.9560 | 3.2068 | 6.7044 | 3.5198 |
| within run % CV | 13.0% | 5.1% | 9.3% | 6.5% | 6.2% |
| within lab % CV | 19.0% | 7.1% | 9.0% | 9.9% | 11.0% |

β-Sitosterol μg/mL

| | | | | | |
|---|---|---|---|---|---|
| Mean (μg/mL) | 0.1020 | 10.2616 | 2.4136 | 6.3204 | 3.0678 |
| within run % CV | 11.7% | 6.3% | 5.1% | 5.4% | 5.6% |
| within lab % CV | 19.4% | 7.2% | 6.1% | 6.3% | 6.5% |

^{a} Acceptance criteria of precision: ≦15% for intra-batch (within run); and ≦20% for inter-batch (within lab).
^{b} LLOQ: lower limit of quantification.
^{c} ULOQ: upper limit of quantification.
^{d} QC: quality control.
^{e} CV: coefficient of variation.

The results of the high-throughput, automated, solid phase extraction sterols/stanols assay were also compared to the results of the manual liquid/liquid extraction (with hexane) sterols/stanols assays. The results demonstrate that the comparison passed the corresponding acceptance criteria known in the art (i.e., mean percent difference: <=+/−20%). Accordingly, the results confirmed and validated the high-throughput, automated sterols/stanols assay as a viable bioanalytical method.

TABLE 4
Comparison of sterol/stanol high-throughput, automated, solid
phase extraction sterols/stanols assay to sterols/stanols assays
measured through manual liquid/liquid extraction method ^{a}

| | Mean Absolute | Mean % |
|---|---|---|
| | Difference | Difference |
| Desmosterol | −0.10 | −12.76% |
| Campesterol | −0.02 | 0.75% |
| Cholestanol | 0.22 | 10.01% |
| β-Sitosterol | 0.04 | 2.29% |

^{a} Acceptance criteria of mean percent difference: < = +/−20%.

### Example 4

### Reference Ranges of Sterols/Stanols High-Throughput Automated Analysis

The rapid, high-throughput automated process, as described in Examples 1 and 2, was performed on 478 patient samples (254 female patients and 234 male patients) to obtain a full reference range (hyper-responder range, optimal-responder range, or hypo-responder range) for desmosterol, campesterol, cholestanol, and β-sitosterol. Each of the sterols/stanols can serve as cholesterol-absorption biomarker and/or cholesterol-synthesis biomarker. These reference ranges include both the ranges of absolute reference levels of the sterol/stanol and the ranges of relative reference levels, using a ratio of the quantity of the sterol/stanol to the quantity of cholesterol, for each sterol/stanol.

The results were sorted based on concentration for each analyte and the approximate quintile ranges and 3 standard deviation (SD) ranges were calculated for each analyte, as shown in Table 5. The resulting reference ranges for desmosterol, campesterol, cholestanol, and β-sitosterol determined by the method of the invention are shown in Table 6.

TABLE 5
Sterol/stanol analysis for reference ranges determination

| | Pt | | | | | | | | | |
|---|---|---|---|---|---|---|---|---|---|---|
| | median | Average | St Dev | Min | Max | −3SD | −2SD | Average | +2SD | +3SD |
| Desmosterol | 0.79 | 0.91 | 0.52 | 0.11 | 4.69 | −0.66 | −0.14 | 0.91 | 1.96 | 2.48 |
| (μg/mL) | | | | | | | | | | |
| Campesterol | 3.10 | 3.42 | 1.90 | 0.53 | 19.60 | −2.28 | −0.38 | 3.42 | 7.23 | 9.13 |
| (μg/mL) | | | | | | | | | | |
| Cholestanol | 2.67 | 2.80 | 1.04 | 0.36 | 10.30 | −0.32 | 0.72 | 2.80 | 4.89 | 5.93 |
| (μg/mL) | | | | | | | | | | |
| Sitosterol | 2.15 | 2.41 | 1.40 | 0.28 | 15.30 | −1.78 | −0.38 | 2.41 | 5.20 | 6.60 |
| (μg/mL) | | | | | | | | | | |
| Desmo | 45.23 | 50.04 | 26.10 | 6.81 | 244.33 | −28.25 | −2.15 | 50.04 | 102.23 | 128.33 |
| ratio, mmol × | | | | | | | | | | |
| 10²/mol | | | | | | | | | | |
| Cholestanol | | | | | | | | | | |
| Camp ratio, | 167.58 | 185.12 | 100.84 | 22.00 | 1106.29 | −117.40 | −16.56 | 185.12 | 386.80 | 487.64 |
| mmol × | | | | | | | | | | |
| 10²/mol | | | | | | | | | | |
| Cholestanol | | | | | | | | | | |
| Cholestanol | 148.51 | 155.85 | 51.13 | 24.17 | 478.83 | 2.45 | 53.58 | 155.85 | 258.12 | 309.25 |
| ratio, mmol × | | | | | | | | | | |
| 10²/mol | | | | | | | | | | |
| Cholestanol | | | | | | | | | | |
| Sito ratio, | 114.02 | 126.48 | 74.36 | 10.16 | 886.99 | −96.58 | −22.23 | 126.48 | 275.20 | 349.55 |
| mmol × | | | | | | | | | | |
| 10²/mol | | | | | | | | | | |
| Cholestanol | | | | | | | | | | |

| | 1st | 2nd | 1st | 2nd | 3rd | 1st | 2nd | 3rd | 4th |
|---|---|---|---|---|---|---|---|---|---|
| | Tertile | Tertile | Quartile | Quartile | Quartile | Quintile | Quintile | Quintile | Quintile |
| Desmosterol | 0.63 | 1.03 | 0.55 | 0.79 | 1.16 | 0.50 | 0.69 | 0.91 | 1.27 |
| (mg/mL) | | | | | | | | | |
| Campesterol | 2.57 | 3.80 | 2.27 | 3.10 | 4.17 | 2.11 | 2.77 | 3.39 | 4.43 |
| (mg/mL) | | | | | | | | | |
| Cholestanol | 2.33 | 2.98 | 2.13 | 2.67 | 3.24 | 2.02 | 2.46 | 2.81 | 3.47 |
| (mg/mL) | | | | | | | | | |
| Sitosterol | 1.75 | 2.66 | 1.55 | 2.15 | 2.97 | 1.43 | 1.93 | 2.41 | 3.17 |
| (mg/mL) | | | | | | | | | |
| Desmo | 37.63 | 53.37 | 33.88 | 45.23 | 59.18 | 31.02 | 39.99 | 50.20 | 64.43 |
| ratio, mmol × | | | | | | | | | |
| 10²/mol | | | | | | | | | |
| Cholesterol | | | | | | | | | |
| Camp ratio, | 139.19 | 203.43 | 124.82 | 167.58 | 223.68 | 114.55 | 153.24 | 186.40 | 239.77 |
| mmol × | | | | | | | | | |
| 10²/mol | | | | | | | | | |
| Cholesterol | | | | | | | | | |
| Cholestanol | 130.14 | 167.36 | 122.18 | 148.51 | 181.47 | 116.87 | 136.87 | 159.73 | 194.27 |
| ratio, mmol × | | | | | | | | | |
| 10²/mol | | | | | | | | | |
| Cholesterol | | | | | | | | | |
| Sito ratio, | 90.88 | 137.37 | 82.52 | 114.02 | 154.96 | 75.83 | 100.42 | 127.02 | 167.85 |
| mmol × | | | | | | | | | |
| 10²/mol | | | | | | | | | |
| Cholesterol | | | | | | | | | |

TABLE 6
Reference ranges of sterols/stanols determined
through the method of the invention
Sterols/Stanol Assay Clinical Reference Range

| Laboratory Test | Hyper Range | Optimal Range | Hypo |
|---|---|---|---|
| Campesterol | >4.43 | 2.11-4.43 | <2.11 |
| (μg/mL) | | | |
| Campesterol Ratio | >239.77 | 114.55-239.77 | <114.55 |
| (10² mmol/mol | | | |
| Cholesterol) | | | |
| Sitosterol (μg/mL) | >3.17 | 1.43-3.17 | <1.43 |
| Sitosterol Ratio | >167.85 | 75.83-167.85 | <75.83 |
| (10² mmol/mol | | | |
| Cholesterol) | | | |
| Cholestanol | >3.47 | 2.02-3.47 | <2.02 |
| (μg/mL) | | | |
| Cholestanol Ratio | >194.27 | 116.87-194.27 | <116.87 |
| (10² mmol/mol | | | |
| Cholesterol) | | | |
| Desmosterol | >1.27 | 0.50-1.27 | <0.50 |
| (μg/mL) | | | |
| Desmosterol Ratio | >64.43 | 31.02-64.43 | <31.02 |
| (10² mmol/mol | | | |
| Cholesterol) | | | |

What is claimed is:

1. A rapid, high-throughput process for analyzing one or more sterols/stanols or derivatives thereof in a plurality of samples, comprising:
introducing a plurality of samples containing one or more sterols/stanols or derivatives thereof into individual vessels in a multi-vessel plate;
cleaving the one or more sterols/stanols or derivatives thereof of each sample in the multi-vessel plate to form sterols/stanols;
extracting the free sterols/stanols of each sample by solid phase extraction; and
detecting the level of the extracted free sterols/stanols in each sample by liquid chromatography tandem mass spectrometry,
wherein the free sterols/stanols do not undergo an additional derivitization step of adding a functional group to the free sterols/stanols prior to the detecting step.

2. The process of claim 1, wherein each vessel is wide and tall enough to allow for adequate mixing, and thin enough to allow the multi-vessel plate to fit in an automated fluid handling station and/or an automated multi-vessel plate handling station.

3. The process of claim 1, wherein the step of cleaving comprises:
pipetting a cleaving agent into each sample in the multi-vessel plate;
vortexing the composition containing the sample and cleaving agent in each vessel; and
heating the multi-vessel plate to a desirable temperature.

4. The process of claim 3, wherein each vessel of the multi-vessel plate is sealed by a matching multi-cap mat.

5. The process of claim 3, wherein the temperature ranges from 40 to 50° C.

6. The process of claim 1, wherein the cleaving step involves hydrolyzing the sterols/stanols or derivatives thereof to form free sterols/stanols.

7. The process of claim 6, wherein the hydrolyzing step involves a saponification of the sterols/stanols derivatives.

8. The process of claim 7, wherein the saponification reaction takes place in presence of an alkali hydroxide or alkaline hydroxide catalyst.

9. The process of claim 8, wherein the catalyst is sodium hydroxide or potassium hydroxide.

10. The process of claim 1, wherein the extracting step comprises:
transferring each sample in the multi-vessel plate to a multi-vessel solid phase extraction plate after the cleaving step; and
eluting the free sterols of each sample from the multi-vessel solid phase extraction plate into a multi-vessel collecting plate.

11. The process of claim 10, wherein the eluting is carried out with dicholoride methane.

12. The process of claim 10, wherein the extracting step further comprises:
drying the eluted free sterols/stanols of each sample in a multi-vessel collecting plate; and
adding a reconstitution solution to the dried free sterols/stanols in the multi-vessel collecting plate to reconstitute the free sterols/stanols.

13. The process of claim 12, wherein the reconstitution solution is a methanol/isopropanol/formic acid solution.

14. The process of claim 1, further comprising:
adding an internal standard to each sample in the multi-vessel plate.

15. The process of claim 14, wherein the internal standard is a deuterated internal standard.

16. The process of claim 1, further comprising:
labeling the plurality of samples in the multi-vessel plate; and
detecting the labeled samples for a sequential processing.

17. The process of claim 16, wherein the labeling step is carried out by an automated bar-coding equipment, and the detecting is carried out by an automated bar code detector.

18. The process of claim 1, wherein the sample is a blood component selected from the group consisting of plasma, serum, red blood cells, whole blood, platelets, white blood cells, sterol/stanol esters, free sterols/stanols, and mixtures thereof.

19. The process of claim 1, wherein the sterols/stanols comprise at least one of desmosterol, campesterol, cholestanol, and sitosterol.

20. The process of claim 1, wherein the sterols/stanols comprise desmosterol, campesterol, cholestanol, and sitosterol.

21. The process of claim 1, wherein the entire process is carried out in less than about 7 minutes for the detecting step.
